(19) United States
(12) Patent Application Publication
(10) Pub. No.: US 2010/0330102 A1
(43) Pub. Date: Dec. 30, 2010

(54) IMMUNOGLOBULIN PREPARATION FOR THE TREATMENT OF HIV-1 INFECTION

(75) Inventors: Daniela CONCAS, Cagliari (IT); Radmila METLAS, Cagliari (IT); Tanja SRDIC, Cagliari (IT)

Correspondence Address: YOUNG & THOMPSON, 209 Madison Street, Suite 500, Alexandria, VA, 22314, US

(73) Assignee: WEZEN BIOPHARMACEUTICALS SRL A SOCIO UNICO, Cagliari (IT)

(21) Appl. No.: 12/492,617

(22) Filed: Jun. 26, 2009

(51) Int. Cl. A61K 39/395; C07K 16/00

(52) U.S. Cl. 424/172.1; 530/389.1; 530/389.6

(57) ABSTRACT

An immunoglobulin (Ig) preparation containing polyclonal IgG-reactive antibodies (Abs), methods for preparing said preparation and pharmaceutical compositions containing it in admixture with suitable excipients, the use of said preparation or composition, alone or in combination with antiretroviral drugs, for the prophylaxis or treatment of HIV-1 infection.

The present invention relates to an immunoglobulin (Ig) preparation for therapeutic use in HIV-1 infection. This Ig preparation contains polyclonal IgG-reactive antibodies (Abs), which have the features of connected Abs. The polyclonal IgG-reactive Abs in this preparation are reactive with IgG coupled to a common matrix used for antibody's affinity purification.

The IgG-reactive Abs are isolated from a fraction of pooled normal human serum (NHS) depleted in IgG, named GammaBind G Flowthrough fraction (GBF fraction), which is capable of reacting with NHS IgG coupled to CNBr-activated Sepharose (IgG-Sepharose), or an equivalent matrix. This IgG-reactive antibody preparation is enriched in IgG₂ monomers and dimers.

The IgG-reactive Abs are separated by exploiting interaction between intact antibody molecules rather than between their F(ab′)2 portions. This approach was chosen because the antibody's constant (C) regions can influence the molecular properties of variable (V) regions, such as the fine specificity interaction between the paratope and the epitope. In particular C regions can affect idiotypes of antibody V regions, i.e. idiotypic network regulation.

Additionally, affinity chromatography was performed at a high ionic strength (5-fold concentrated PBS) in order to reduce the interaction of low-affinity antibody with IgG-Sepharose. Such experimental conditions may be responsible for the observed enrichment in the IgG₂ subclass as well as for the low level of rheumatoid factor (RF).

The IgG-reactive Abs described in this invention potently neutralize in vitro infection of peripheral blood lymphocytes (PBL) by HIV-1.

Another aspect of the invention concerns the use of the Ig preparation according to the invention as a medicinal product.

### BACKGROUND OF THE INVENTION

The development of an effective vaccine is the most promising approach to contain the ongoing HIV pandemic [1]. However, an immunogen able to elicit broadly neutralizing Abs is yet to be identified. A conserved cluster of carbohydrate residues of gp120 might be a good vaccine candidate [2], but these epitopes are present on many extracellular host proteins and increased levels of Abs of high affinity and broad specificity against carbohydrates may cause autoimmune conditions [3-5]. In healthy individuals, a functional immune network embodies a wide range of natural autoantibodies, with T and B lymphocyte clones being essential in self-assertion [6]. Autoimmune disorders can result from deviation of normal patterns of immunoglobulin (Ig) connectivity [7, 8]. Recent data showed that broadly and potently neutralizing anti-HIV-1 human monoclonal Abs made from B cells of HIV-1 infected individuals, but rarely present in HIV-1 subjects, have features of natural autoantibodies [4, 9]. This suggests that broadly neutralizing Abs might be generated in the context of disrupted immunoregulation.

Natural antibodies maintain the immune homeostasis in healthy individuals by the regulation of antibody and of T cell response via network-like interactions [10]. In addition, natural antibodies act as an early defence mechanism against bacteria and viruses [11]. Several lines of evidence demonstrate that antibodies directed against carbohydrate moieties of viral glycoproteins, including those of HIV-1 gp120, are present in normal human serum (NHS) [12-14]. Recently, we reported that affinity chromatography-separated IgG-reactive antibodies (then named anti-IgG antibodies) from normal human serum prevent human peripheral blood mononuclear cell (PBMC) infection by HIV-1. Conversely, unfractionated total NHS IgG failed to neutralize HIV-1 infectivity even if the concentration of the unfractionated IgG was three-fold higher than that of IgG-reactive antibodies [15]. Previous data showed that affinity chromatography separated IgG-reactive antibodies also react with gp120 V-3 loop derived peptide [16,17]. It has been postulated that HIV-1 may interfere with the immune network because of sequence similarity between gp120 and immunoglobulins (Igs) [18].

IgG-reactive antibodies purified from NHS in addition to predominant IgG also contain some IgM and IgA. Since GammaBind G Sepharose (GB) binds all IgG isotypes, we attempted to separate IgG-reactive IgM and IgA antibodies from the GB flowthrough fraction (GBF) depleted in IgG. Unexpectedly we observed that a low but reproducible amount of IgG was not retained by the GammaBind G Sepharose, a high percent of which was purified as IgG-reactive antibodies by affinity purification on CNBr-activated Sepharose coupled with IgG (IgG-Sepharose).

The IgG-reactive antibodies purified from the GBF fraction contain not only IgG but also IgM and IgA. As shown in Table 1, IgG in IgG-reactive GBF Abs represents 20% of the total Igs whereas IgG accounts for about 70% in IgG-reactive NHS Abs.

The IgG-reactive antibodies described in this invention are enriched both in IgG₂ monomers and in IgG₂ dimers (Table 2 and FIG. 1). IgG₂ is a subclass generally accepted to be produced in response to polysaccharides (PS) [19]. The presence of IgG₂ dimers in the preparation is consistent with the recent discovery that human sera contain covalent IgG₂ dimers [20].

Very recent data have shown the importance of the natural anti-HIV immune response in HIV-infected individuals [21, 22].

The impact of IgG isotypes on HIV-1 neutralization is still a matter of discussion but important for designing an effective passive immunization against HIV-1 in humans, in particular because total IgG₂ level in HIV-1-infected subjects was found to be lower than in the healthy controls [23, 24]. According to several authors, the contribution of IgM in HIV-1 neutralization is low due to low-affinity [25, 26]. Compared to IgG, the neutralizing activity of IgA is less important [27]. However, polymeric IgA is regarded as the main immunological barrier at mucosal sites [28].T

The presence of a relatively low amount of IgG in the GBF fraction indicates that this specific IgG may have particular features relating to the CH2-CH3 interface region of the Fc portion which interacts with IgG-binding domains of GammaBind G protein [29]. We suggest that the IgG isotypes present in the GBF fraction may share similarities with autophylic antibodies. These rare antibodies are capable of forming self-complexes [30] and interact via the self-binding domain of the unconventional binding sites otherwise used by superantigens such as gp120 [31].

The presence of IgG₂ covalent dimers detected in the preparation (FIG. 1B) may increase the antigenic avidity of IgG₂, as well as its interaction with FcγRIIa. Furthermore, according to recent data, HIV-1 specific CD4 Th1 cell responses combined with IgG₂ antibodies are the best predictor for persistence of long-term non-progression [32].

Since V-region-identical antibodies differing in the C region demonstrate differences in fine specificity and idiotype, thus regulating immunity and tolerance to Ids (33, 34), this study was performed on intact Ab molecules instead of their F(ab′) fragments. Alterations in Ids following isotype change could have major implications on several current immunological concepts as well as on the generation of Abs to certain Ags and on the isotype preference observed in Abs to polysaccharides (35).

In this invention we present evidence that IgG-reactive antibodies from the GBF fraction contain an IgG₂/IgG₁ ratio of almost 2, which is the inverse of that found in NHS. In addition, we provide evidence that this antibody preparation is highly efficient in HIV neutralization.

The IgG-reactive antibody preparation described in the present invention displays neutralizing activity both against a laboratory-adapted strain and against primary isolates (FIG. 2, FIG. 3, and Table 3).

As long as difficulties in developing an effective anti-HIV vaccine remain unresolved, current data suggest that passive immunization with IgG-reactive Ab preparations, enriched in IgG₂ monomers and dimers, able to block virus infectivity and potentially capable of re-establishing immune homeostasis, could prove beneficial in the treatment of HIV-1 infection and as post-exposure prophylaxis.

In summary, an NHS fraction has been identified which exhibits particular features,—namely interaction with other Igs, IgG₂ prevalence (in particular of IgG₂ dimers), and the ability to neutralize in vitro HIV-1 infection. This fraction may play a role in natural protection against HIV-1 infection. It is suggested that IgG-reactive antibodies from the GBF fraction alone or in combination with IgG-reactive IgG obtained from total NHS IgG by affinity chromatography on IgG-Sepharose (designated as IgG-reactive IgG), has therapeutic potential in HIV-1 infected individuals.

### PRIOR ART

CA1341505 relates to a polyclonal immunoglobulin-based intravenous preparation for the therapy and prophylaxis of bacterial infections, which is enriched in IgM.

U.S. Pat. No. 6,932,969 discloses a method for preparing Ig fractions from human polyvalent IVIg which comprise natural antibodies and contain IgG which exhibit interactions of the idiotypic type with one another.

US2003/0077841 discloses a method of enhancing the activity of an immunoglobulin preparation, comprising at least one step of denaturing and subsequently renaturing the immunoglobulin.

WO02/072637 discloses human antibodies against IgG total fraction, capable of neutralizing HIV-1 and obtainable by subjecting sera from normal, non HIV-infected subjects to affinity chromatography using resins bound to human IgG total fraction, and the use thereof for the preparation of a medicament for the treatment of HIV infection.

Unlike the cited prior documents, the present invention describes connected antibodies denoted as IgG-reactive GBF Abs, which are present in NHS IgG, are recovered in the GBF fraction and are purified by affinity chromatography using their reactivity with intact total NHS IgG.

The IgG-reactive Ab preparation according to the invention neutralize HIV-1 infection. The observed neutralizing activity was obtained by pre-incubating the Abs with the cells prior to adding virus to the assay. In contrast, previous neutralization data reported in WO02/072637, in the name of the same applicants, were obtained by pre-incubating an IgG-reactive antibody preparation from NHS with the virus preparation prior to adding the cells. Without being bound by theory, it is possible that the mechanism of neutralization for the IgG-reactive preparation according to the present invention could involve interaction of IgG-reactive antibodies from the GBF fraction with host cell-derived molecules incorporated by budding HIV-1 virions.

Furthermore, the presence of the IgG₂ covalent dimers detected in the IgG-reactive GBF Ab preparation (FIG. 1) may increase the antigenic avidity of IgG₂, as well as its interaction with Fc receptors involved in the control of B cell activity and in dendritic cells (DC) maturation and differentiation

### DESCRIPTION OF THE INVENTION

A normal human serum (NHS) Ig fraction is identified which exhibits interaction with other Igs involving intact antibody molecules under high ionic strength conditions (5-fold concentrated PBS); is enriched in IgG₂, and in particular in IgG₂ dimers; has low RF activity; and displays neutralizing activity against HIV-1.

This IgG-reactive Ab fraction enriched in IgG₂, which represents a first aspect of the invention, is prepared from normal human serum—i.e. serum from healthy individuals—through a process which comprises the following steps:
(a) passing NHS through a matrix specifically reactive with human IgG, particularly GammaBind G Sepharose, and recovering the matrix bound IgG fraction and the matrix-unbound fraction or “GammaBind G flow-through” (GBF) fraction;
(b) eluting the matrix-bound IgG fraction obtained in (a), to obtain affinity purified IgG;
(c) covalent binding of the affinity-purified IgG obtained in (b), to CNBr-activated Sepharose 4B, or to an equivalent matrix, to obtain IgG-Sepharose;
(d) contacting the GBF fraction obtained in (a) with IgG-Sepharose in 5-fold concentrated PBS and eluting the IgG-Sepharose bound Igs to obtain IgG-reactive GBF Abs.

As used herein, 5-fold concentrated PBS indicates the following buffer composition: 5×(8 g NaCl, 0.2 g KCl, 1.44 g Na₂HPO₄, 0.24 g KH₂PO₄ in 1000 ml distilled H₂O at pH 7.4 adjusted with HCl).

In a preferred embodiment, the IgG-reactive GBF Abs (step (d)) are combined into a single preparation with IgG-reactive IgG from NHS which are obtained through a process which comprises the following steps:

(a′) passing NHS through a matrix able to bind human IgG, particularly GammaBind G Sepharose matrix, and eluting the matrix-bound IgG fraction to obtain affinity purified IgG;

(b′) covalent binding of one aliquot of affinity purified IgG obtained in (a′), to CNBr-activated Sepharose 4B or an equivalent matrix, to obtain IgG-Sepharose;

(c′) contacting a second aliquot of affinity purified IgG obtained in (a′), with IgG-Sepharose in 5-fold concentrated PBS obtained in (b′), and eluting IgG-Sepharose bound Igs to obtain IgG-reactive IgG.

In a preferred embodiment, the total human IgG is prepared by affinity chromatography of normal human serum on GammaBind G Sepharose, by loading normal human serum on GammaBind G Sepharose and eluting with an acid solution which is preferably 0.5 M acetic acid, pH 3.

In a further preferred embodiment, the total human IgG-coupled resin is prepared by attaching the affinity-purified total human IgG to CNBr-activated Sepharose and is referred to as IgG-Sepharose.

In a yet further preferred embodiment, the elution of the GBF antibodies bound to IgG-Sepharose is carried out with an acid solution, which is preferably 0.1 M citrate, pH 2.5.

In a yet further preferred embodiment, the IgG-reactive GBF antibodies present a rheumatoid factor activity not exceeding by more than 10% a cut off level of 25 U/ml, as determined by commercially available ELISA, ORG 522S Rheumatoid Factor Screen (GTI, Brookfield, Wis.).

The IgG present in the GBF fraction represents about 0.3% of the total IgG present in NHS. This indicates that not all of the IgGs present in NHS are bound by GammaBind G Sepharose. The eluted IgG is efficiently recovered by affinity purification on IgG-Sepharose, as set out above. In addition to residual IgG, the GBF fraction contains IgM and IgA isotypes.

IgG subclass analysis of the IgG-reactive antibodies separated from NHS (IgG-reactive IgG) and the GBF fractions (IgG-reactive GBF antibodies) by specific IgG subclass ELISA assay showed that there is considerable enrichment in IgG₂ compared to IgG₁ in IgG-reactive IgG and in IgG-reactive GBF antibodies. Western blot analysis confirmed the abundance of IgG₂ and also showed the presence of IgG₂ dimers (FIG. 1). IgG₂ is the main IgG subclass generated by humans in response to polysaccharide antigens. IgG₂ deficiency is frequently associated with an increased susceptibility to infection, therefore its replacement by means of the IgG-reactive Ab preparation of the invention will be beneficial in the prevention or treatment of infectious diseases.

In particular, the IgG-reactive antibody preparation of the invention was found to neutralize HIV-1 infection. In a further embodiment, therefore, the invention provides a method of post-exposure prophylaxis or treatment of HIV-1 infection which consists in administering to a subject in need thereof an antibody preparation as herein provided. A therapeutically effective daily dose of IgG-reactive GBF antibodies or combined IgG-reactive GBF antibodies and IgG-reactive IgG will be about 10 mg/kg body weight.

The antibodies may be administered to a subject infected by HIV or at risk of developing HIV infection post-exposure to the virus.

Due to their HIV-1 neutralization properties, the IgG-reactive antibodies preparation described in this invention may be conveniently used to improve the anti-HIV-1 activity of other immunoglobulin preparations, especially of intravenous immunoglobulin (IVIg), which is a therapeutic preparation of normal human polyclonal IgG obtained by pooling plasma from a large number of healthy blood donors.

For the therapeutic use, the antibody preparation according to the invention may be formulated with pharmaceutically acceptable excipients such as buffering agents, stabilizing agents, solubilizers, diluents, isotonicity agents and the like.

The subcutaneous administration route is preferred. However, the preparation can also be administered via the intravenous or intramuscular routes.

### DESCRIPTION OF THE DRAWINGS

FIG. 1. Western blot data and densitometric quantitation data (representative blot)

A: Western blotting analysis was performed by methods that allow detection of IgG₂ monomers and dimers.

Lane 1—affinity purified IgG (10 μg/well); Lane 2—affinity purified IgG (4 μg/well); Lane 3—IgG-reactive Abs from NHS (4 μg/well); Lane 4—IgG-reactive Abs from NHS (2 μg/well); Lane 5—IgG-reactive Abs from the GBF fraction (2 μg/well).

B: Densitometric quantitation of the Western blot bands is presented as the percentage of the IgG₂ dimers of the sum of integrated density (ID) values of IgG₂ monomers and dimers. Results are presented as a mean±SD from three independent experiments. Quantification of the blots was performed using a GS-710 Calibrated imaging densitometer (Bio-Rad) and the Image Quant software program.

FIG. 2. Neutralization capacity of IgG-reactive Abs from the GBF fraction against infection of PBL by HIV-1_{BaL} strain

The assay is a representative of three independent experiments (each point is the mean±standard error for four independent replicates).

FIG. 3. Neutralization capacity of IgG-reactive Abs from the GBF fraction against infection of PBL by HIV-1_{JR-CSF} primary isolate.

The assay is a representative of three independent experiments (each point is the mean±standard error for four independent replicates).

### EXAMPLE

Serum Samples

Normal human serum from healthy donors was collected according to internal standard operating procedures. Serum was pooled from 10 to 20 donors. The serum pool consisted of equal volumes of each donor's serum (usually 3 ml from each donor were used). Prior to affinity chromatography, serum was heat inactivated at 56° C. for 30 min and then filtered through a 0.2 μm-pore size Millipore filter.

Preparation of GammaBind G Sepharose Flow-Through fraction (GBF Fraction)

2 ml of normal human serum (NHS), i.e. about 18.0 mg of IgG, was dialysed by centrifugation (MW limit 50K Da) in a binding buffer, and then was loaded into a 2.0 ml GammaBind G Sepharose column. The column capacity was 18 mg of human IgG per 1 ml of drained gel. The GBF fraction thus obtained was dialysed and concentrated by centrifugation in PBS before being used for measurement of Ig class concentration by capture ELISA. GBF fractions with IgG concentrations of at least 10 μg per ml were used for the separation of IgG-reactive Abs.

Affinity Chromatography and Capture ELISA

Separation of IgG-reactive antibodies from total NHS IgG (affinity purified on GammaBind G Sepharose), and from the GBF (GammaBind G Flowthrough) fraction was performed on IgG-Sepharose (IgG-coupled to CNBr-activated Sepharose 4B), as presented in the above disclosure of the invention. Samples of affinity purified IgG-reactive Abs obtained from six independent runs were concentrated by ultrafiltration (YM-50, cut-off MW 50,000). Ig concentration was adjusted to 100 μg per ml for the IgG-reactive antibody preparations obtained both from total NHS IgG and from the GBF fraction. Obtained IgG-reactive antibodies were tested for the presence of IgG₂ monomers and dimers, RF (Rheumatoid Factor) activity and for the ability to neutralize HIV-1 infection in vitro.

It should be noted that the concentration of IgG-reactive GBF antibodies was expressed as that of the total Ig content and thus, represents the sum of all isotypes. Two preparations (denoted as “Prep 1” and “Prep 2”) of IgG-reactive GBF Abs obtained from two different serum pools were used in some of the virus neutralization assays. These preparations differed in the yield and proportional recovery of IgG, IgM and IgA. However, the magnitude of such differences was consistent with normal experimental variation.

Analysis of IgG Subclasses

The concentrations of the individual IgG subclasses were measured by PeliClass™ human IgG subclass ELISA kit (CLB, Amsterdam) according to the manufacturer's instructions. IgG subclass concentration of IgG-reactive GBF antibodies was expressed as μg/ml of serum used for Ig separation.

SDS-PAGE and Western Blotting

To examine heavy (H) and light chains (L), the IgG-reactive Abs obtained from the GBF fraction and the IgG purified on GammaBind G Sepharose were analyzed by reducing SDS-PAGE on 10% gel. IgG monomers and dimers were analyzed by denaturing but non-reducing SDS-PAGE on 4% Tris-glycine gels as described previously [20].

The SDS-PAGE-resolved antibodies were electrophoretically transferred to a nitrocellulose membrane. Nonspecific sites were blocked by incubating the membrane for 1 h at room temperature in PBS containing 0.2% (v/v) Tween 20 and 5% (w/v) nonfat dried milk. For immunodetection, mouse mAbs specific for human γ1 and γ2 were used as a primary reagent for overnight incubation at 4° C. After washing, the blot was probed with peroxidase-conjugated goat anti-mouse IgG for 1 h at room temperature. Detection was performed using the Supersignal Substrate system (Pierce) (as recommended by the manufacturer).

Virus Neutralization Assays

Peripheral blood lymphocytes (PBL) from uninfected donors were prepared by Ficoll-Hypaque density centrifugation from heparinised blood. These fresh PBL, at a concentration of 2×10⁶ cells per ml, were stimulated for 2 days with 5 μg of PHA per ml in RPMI medium containing 20 U/ml of recombinant interleukin-2, 20% fetal calf serum, 2% penicillin-streptomycin and 1% L-glutamine. Cultures were carried out in tissue culture flasks incubated in 5% CO₂ at 37° C. After 3 days, the cells were readjusted to a concentration of 2×10⁶ cells per ml in the medium described above, except that the medium lacked PHA and was supplemented with 20 U/ml of recombinant interleukin-2.

A defined volume of stimulated PBL cells (2×10⁶ cells per ml) was incubated with each of the following six test items,—with three different concentrations of IgG-reactive Abs separated from the GBF fraction (2 μg/ml, 0.6 μg/ml and 0.3 μg/ml), with anti-CCR5 Ig (10 μg/ml) [22], with mAb 2G12 [3] as positive control (10 μg/ml), and with medium alone as negative control, for 30 min at 37° C. before adding virus. Concentrations of 0.5 ng/ml of HIV-1_{BaL} strain, of HIV-1_{NDK} (X4-tropic isolate) and of HIV-1_{JR-CSF} (R5-tropic isolate) were used. Incubation was continued for 3 h at 37° C. in 5% CO₂. The cells were then washed and cultured in RPMI medium supplemented with 20 U/ml of recombinant interleukin-2 and 10% fetal calf serum. After 3 or 6 days of culture, when the p24 concentration was between 750 and 1000 ng/ml, the supernatants were collected and the virus was lysed by Triton X-100. The supernatants were then quantified for viral production by measuring p24 release using a p24 antigen capture ELISA (HIV-1 core profile ELISA, Dupont de Nemours, Les Ullis, France). The percent neutralization was calculated as [1−(mean value of p24 in the presence Abs/mean value of p24 in the absence of Abs)]×100.

Results

Characterization of IgG-Reactive Antibodies

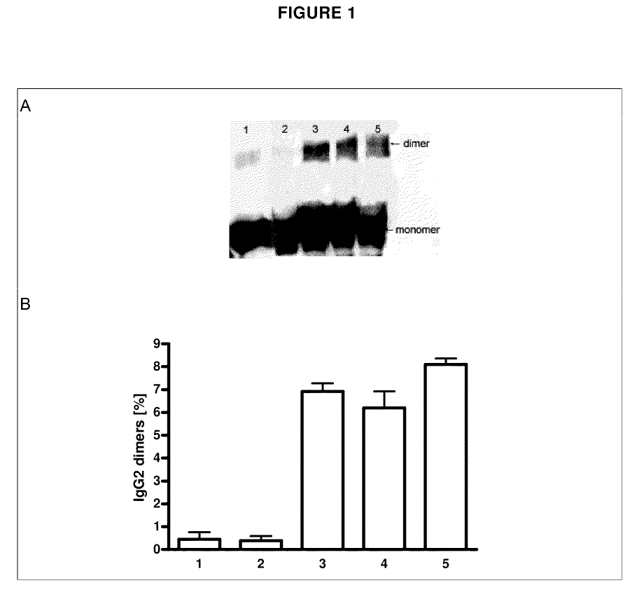

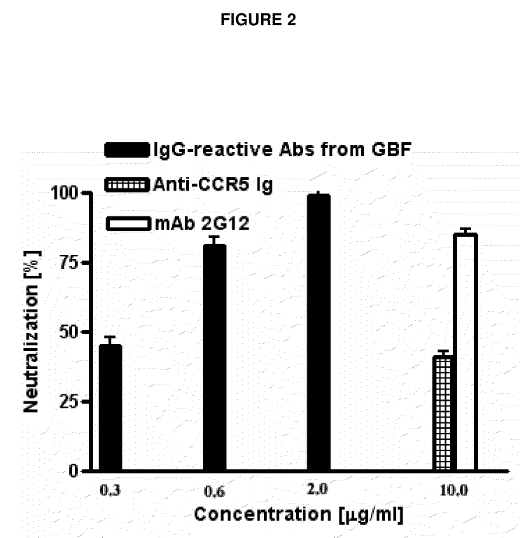

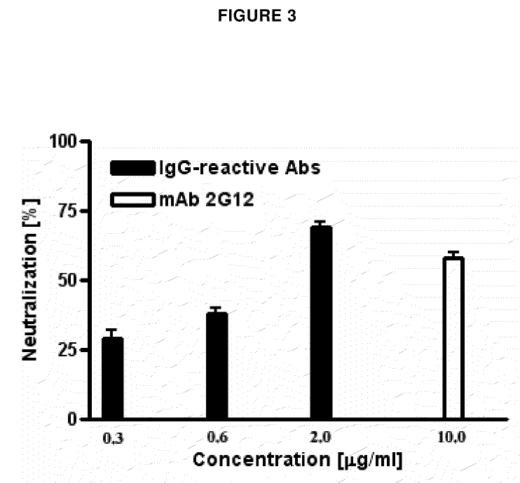

Evaluation by reducing SDS-PAGE of the IgG purified on GammaBind G Sepharose (GB) used for coupling to CNBr-activated Sepharose (denoted as IgG-Sepharose) revealed no contamination with other Ig isotypes (FIG. 1A, Lane 3). A comparative analysis, by Ig class, of IgG-reactive antibodies purified on IgG Sepharose recovered from pooled NHS and from the GB Flowthrough (GBF) fraction is shown in Table 1. The proportion of IgG, IgM and IgA isotypes recovered from NHS on IgG-Sepharose as IgG-reactive antibodies was 0.33%, 0.76% and 0.21% of the loaded Ig isotypes, respectively (Table 1). For the GBF fraction, the proportion of each of the loaded IgG, IgM and IgA isotypes which were recovered as IgG-reactive antibodies was 15%, 1.65% and 0.45%, respectively (Table 1). Some decrease in IgM and IgA concentration in the GBF fraction, compared to NHS, was noted. When the concentration of loaded IgG was lower, as was the case for the GBF fraction compared to NHS, the proportional recovery of IgG-reactive IgM and IgA was greater for the GBF fraction (1.65% and 0.45%, respectively) than that from NHS (0.76% and 0.21%, respectively) (Table 1). One explanation might be that IgG has the highest affinity for IgG-Sepharose. Small variations in the yield and proportional recovery of different Ig isotypes between different serum pools were observed.

TABLE 1
IgG-reactive Abs purified from NHS and from the
GammaBind G Sepharose Flowthrough (GBF) fraction: comparison
between NHS and GBF, of yield and of proportional recovery,
for each Ig class.

| | | | |
|---|---|---|---|
| | | Purified | |
| | | IgG-reactive | |
| | Loaded | Abs | |
| | (mg/ml of | (μg/ml | Recovery |
| Ig | serum) | of serum) | (%) |

| Class | NHS | GBF | NHS | GBF | NHS | GBF |
|---|---|---|---|---|---|---|
| IgG | 6.50 | 0.02 | 21.00 | 3.00 | 0.33 | 15.00 |
| IgM | 0.38 | 0.23 | 2.90 | 3.80 | 0.76 | 1.65 |
| IgA | 2.30 | 1.57 | 4.78 | 7.00 | 0.21 | 0.45 |

The reducing SDS-PAGE, performed for IgG-reactive antibodies obtained from NHS on IgG Sepharose, revealed, in accordance with the data obtained by Capture ELISA relative to isotype-matched standards (Table 1), a predominant content of IgG and a weak band corresponding to μ heavy chain (FIG. 1A, Lane 2).

Monomers and dimers were observed in IgG-reactive Abs obtained from NHS (FIG. 1B, Lane 2 and 3) and in those obtained from the GBF fraction (FIG. 1B, Lane 4), using anti-human γ2-specific Ab. However, no bands corresponding to IgG₂ dimers were observed in GammaBind G Sepharose purified IgG, for the amount applied (FIG. 1B, Lane 1). Also, no dimers were found in the IgG₁ subclass by Western blotting using γ1-specific Abs, either for NHS-derived IgG-reactive antibodies (FIG. 1C, Lane 2), for GBF fraction IgG-reactive antibodies (FIG. 1C, Lane 3), or for purified IgG (FIG. 1C, Lane 1). The data presented also indicate that the band migrating at the position for monomer IgG₁ was most prominent for affinity purified IgG (FIG. 1C, Lane 1), in comparison to the IgG-reactive Abs (FIG. 1C, Lane 2 and 3).

IgG subclass distribution in IgG-reactive Abs obtained from NHS and in those obtained from the GBF fraction, measured by quantitative ELISA, is presented in Table 2. These data revealed that in contrast to the characteristic dominance of IgG₁ subclass in pooled NHS, the IgG-reactive antibodies obtained from NHS and from the GBF fraction each showed a dominance of IgG₂. Thus, a higher level of IgG₂ is a characteristic of IgG-reactive Abs irrespective of their source.

TABLE 2
IgG subclasses distribution of IgG-reactive antibodies.

| | | | | IgG-reactive | IgG- |
|---|---|---|---|---|---|
| | PeliClass | | | Abs from | reactive |
| | (mg/ml) | | Pooled | NHS, | Abs from |
| IgG | Control | Reference | (NHS) | mean ± SD, | GBF |
| subclass | serum | ranges | (mg/ml) | (μg/ml) | (μg/ml) |
| IgG₁ | 6.33 | [4.9-11.40] | 8.91 | 4.28 ± 0.01 | 1.4 |
| IgG₂ | 4.69 | [1.50-6.40] | 5.70 | 11.16 ± 1.49 | 2.61 |
| IgG₃ | 0.52 | [0.20-1.10] | 0.77 | 0.62 ± 0.12 | 0.16 |
| IgG₄ | 0.66 | [0.08-1.40] | 1.05 | 1.06 ± 0.31 | 0.22 |

Reference ranges for IgG subclasses in healthy individuals were from PeliClass™ ELISA kit product information. IgG subclass content is presented for the pooled normal human serum (NHS) from which the IgG-reactive Abs were separated. The concentration of IgG-reactive Abs for each IgG sub-class is given per ml of serum from which the antibodies were separated. For IgG-reactive antibodies from NHS, values are presented as mean±SD of three independent experiments, and for IgG-reactive antibodies from the GBF fraction, as the mean of two experiments

IgG-Reactive Antibodies from the GBF Fraction Prevent Infection of PBL by HIV-1_{BaL} and by the Primary Isolates HIV-1_{JR-CSF} and HIV-1_{NDK}.

Virus neutralization assays were performed with the HIV-1_{BaL} strain and the primary isolates HIV-1_{JR-CSF} and HIV-1_{NDK}. FIG. 2 and FIG. 3 display the neutralizing activity of IgG-reactive antibodies from the GBF fraction against HIV-1_{BaL} and HIV-1_{JR-CSF} infection of PBL, respectively. The results of the neutralization assays are expressed as percent reduction in p24 release. The inhibition of infectivity by IgG-reactive antibodies isolated from the GBF fraction is dose-dependent (FIG. 2 and FIG. 3). There is almost complete virus neutralization at 2 μg/ml, while at 0.3 μg/ml inhibition of infectivity is comparable to that by 10 μg/ml of anti-CCR5 Ig (FIG. 2). This natural antibody to CCR5, affinity purified from IVIg, is known to be able to block HIV-1 infection at the concentration tested [22]. Virus neutralization by IgG-reactive antibodies was comparable to that by the mAb 2G12, which was tested at a concentration of 10 μg/ml only (FIG. 2 and FIG. 3). The concentration of mAb 2gG12 was selected based on IC50 and IC90 data obtained in our laboratory being valid for most clades tested. The mAb 2G12 is a broadly and potently neutralizing human monoclonal antibody which is directed against a unique carbohydrate-dependent epitope on gp120 and which inhibits gp120 interaction with CCR5 [3].

IgG-reactive antibodies obtained from the GBF fraction of two different pools of NHS (Prep 1 and Prep 2), were tested for their ability to neutralize HIV-1_{NDK} and HIV-1_{JR-CSF}. As shown in Table 3, the IC50 of Prep 1 and of Prep 2 for HIV-1 NDK was 0.6 μg/ml and 1.8 μg/ml, respectively, and the IC50 of Prep 1 and of Prep 2 for HIV-1_{JR-CSF} was 0.7 μg/ml and 0.4 μg/ml, respectively. These data indicate a low variation in neutralization activity between different preparations.

TABLE 3
IC50 of two different preparations of IgG-reactive
antibodies from the GBF fraction for HIV-1_{NDK} and HIV-1_{JR-CSF}.

| | |
|---|---|
| | IC ≦ 90 |
| IC 50 | mAb |

| | Prep. 1 | Prep. 2 | 2G12 |
|---|---|---|---|
| Primary isolate | (μg/ml) | (μg/ml) | (μg/ml) |
| HIV-1_{NDK} (X4- | 0.6 | 1.8 | 10 |
| tropic) | | | |
| HIV-1_{JR-CSF} (R5- | 0.7 | 0.4 | 10 |
| tropic) | | | |

Concentrations (μg/ml) of IgG-reactive antibodies from two different preparations (Prep 1 and Prep 2) which inhibit p24 release by 50% relative to cultures without antibodies.

### REFERENCES

[1] Letvin N L. Progress in the development of an HIV-1 vaccine. Science 1998; 280: 1875-1880.
[2] Calarese D A, Lee H K, Huang C Y, et al. Dissection of the carbohydrate specificity of the broadly neutralizing anti-HIV-1 antibody 2G12. Proc Natl Acad Sci USA 2005; 102: 13372-13377.
[3] Sanders R W, Venturi M, Schiffner L, et al. The mannose-dependent epitope for neutralizing antibody 2G12 on human immunodeficiency virus type 1 glycoprotein gp120. J Virol 2002; 76:7293-7305.
[4] Haynes B F, Fleming J, St Clair E W, Katinger H, Stiegler G, Kunert R, et al. Cardiolipin polyspecific autoreactivity in two broadly neutralizing HIV-1 antibodies. Science 2005; 308: 1906-1908.
[5] Werdelin O, Meldal M, Jensen T. Processing of glycans on glycoprotein and glycopeptide antigens in antigen-presenting cells. Proc Natl Acad Sci USA 2002; 99: 9611-9613.
[6] Coutinho A. Beyond clonal selection and network. Immunol Rev 1989; 110: 63-87.
[7] Lundkvist I, Coutinho A, Varela F, Holmberg D. Evidence for a functional idiotypic network among natural antibodies in normal mice. Proc Natl Acad Sci USA 1989; 86: 5074-5078.
[8] Padierna-Olivos L, Moreno-Altamirano M M, Sanchez-Colon S, Masso-Rojas F, Sanchez-Garcia F J. Connectivity and HIV-1 infection: role of CD4(+) T-cell counts and HIV-1 RNA copy number. Scand J Immunol 2000; 52: 618-627.
[9] Meffre E, Milili M, Blanco-Betancourt C, Antunes H, Nussenzweig M C, Schiff C. Immunoglobulin heavy chain expression shapes the B cell receptor repertoire in human B cell development. J Clin Invest 2001; 108: 879-886.
[10] Coutinho A, Kazatchkine MD, Avrameas S. Natural autoantibodies. Curr Opin Immunol 1995; 7: 812-818.
[11] Ochsenbein A F, Fehr T, Lutz C, et al. Control of early viral and bacterial distribution and disease by natural antibodies. Science 1999; 286: 2156-2159.
[12] Tomiyama T, Lake D, Masuho Y, Hersh E M. Recognition of human immunodeficiency virus glycoproteins by natural anti-carbohydrate antibodies in human serum. Biochem Biophys res Commun 1991; 177: 279-285.
[13] Arendrup M, Hansen J E, Clausen H, Nielsen C, Mathiesen L R, Nielsen J O. Antibody to histo-blood group A antigen neutralizes HIV produced by lymphocytes from blood group A donors but not from blood group B donors. AIDS 1991; 5: 441-444.
[14] Preece A F, Strahan K M, Devitt J, Yamamoto F, Gustafsson K. Expression of ABO or related antigenic carbohydrates on viral envelopes leads to neutralization in the presence of serum containing specific natural antibodies and complement. Blood 2002; 99: 2477-2482.
[15] Metlas R, Srdic T, Veljkovic V. Anti-IgG antibodies from sera of healthy individuals neutralize HIV-1 primary isolates. Curr HIV Res 2007; 5: 261-265.
[16] Metlas R, Trajkovic D, Srdic T, Veljkovic V, Colombatti A. Human immunodeficiency virus V3 peptide-reactive antibodies are present in normal HIV-negative sera. AIDS Res Hum Retroviruses 1999; 15: 671-677.
[17] Metlas R, Trajkovic D, Srdic T, Veljkovic V, Colombatti A. Anti-V3 and anti-IgG antibodies of healthy individuals share complementarity structures. J Acquir Immune Defic Syndr 1999; 21: 266-270.
[18] Metlas R. Veljkovic V. Does the HIV-1 manipulate immune network via gp120 immunoglobulin-like domain involving V3 loop? Vaccine 1995; 13: 355-359.
[19] Barrett D J, Ayoub E M. IgG₂ subclass restriction of antibody to pneumococcal polysaccharides. Clin Exp Immunol 1986; 63:127-134.
[20] Yoo E M, Wims L A, Chan L A, Morrison S L. Human IgG2 can form covalent dimers. J Immunol 2003; 170: 3134-3138.
[21] Scheid J F, Mouquet H, Feldhahn N, et al. Broad diversity of neutralizing antibodies isolated from memory B cells in HIV-infected individuals. Nature 2009. doi: 10.1038/nature07930
[22] Bouhlal H, Hocini H, Quillent-Gregoire C, et al. Antibodies to C—C chemokine receptor 5 in normal human IgG block infection of macrophages and lymphocytes with primary R5-tropic strains of HIV-1. J Immunol 2001; 166: 7606-7611.
[23] Cummins L M, Weinhold K J, Matthews T J, et al. Preparation and characterization of an intravenous solution of IgG from human immunodeficiency virus-seropositive donors. Blood 1991; 77: 1111-1117.
[24] Raux M, Finkielsztejn L, Salmon-Céron D, et al. IgG subclass distribution in serum and various mucosal fluids of HIV type 1-infected subjects. AIDS Res Hum Retroviruses Apr. 10, 2000;16 (6):583-94.
[25] Llorente M, Sanchez-Palomino S, Manes S, et al. Natural human antibodies retrieved by phage-display libraries from healthy donors: polyreactivity and recognition of human immunodeficiency virus type 1 gp120 epitopes. Scand J immunol 1999; 50: 270-279.
[26] Toràn J L, Kremer L, Sanchez-Pulido L, et al. Molecular analysis of HIV-1 gp120 antibody response using isotype IgM and IgG phage display libraries from a long-term non-progressor HIV-1 infected individual. Eur J Immunol 1999; 29: 2666-2675.
[27] Burrer R, Salmon-Ceron D, Richert S, et al. Immunoglobulin G (IgG) and IgA, but also nonantibody factors, account for in vitro neutralization of Human Immunodeficiency Virus (HIV) type 1 primary isolates by serum and plasma of HIV-infected patients. J Virol 2001; 75: 5421-5424.
[28] Wright A, Lamm M E, Huang Y T. Excretion of Human Immunodeficiency Virus type 1 through polarized epithelium by immunoglobulin A. J Virol 2008; 82:11526-35.
[29] Kato K, Lian L Y, Barsukov I L, et al. Model for the complex between protein G and antibody Fc fragment in solution. Structure 1995; 15: 79-85.
[30] Kohler H. Superantibodies-synergy of innate and acquired immunity. Applied Biochemistry and Biotechnology 2000; 83:
[31] Karry S, Zouali M. Identification of the B cell superantigen binding site of HIV-1 gp120. Proc Natl Acad Sci USA 1997; 94: 1356-1360
[32] Martinez V, Costagliola D, Bonduelle O, et al. Combination of HIV-1-specific CD4 Th1 cell responses and IgG2 antibodies is the best predictor for persistence of long-term nonprogression. J Infect Dis 2005; 191: 2053-2063.
[33] Reitan S K and Hannestad K. Immunoglobulin heavy chain constant regions regulate immunity and tolerance to idiotypes of antibody variable regions. Proc Natl Acad Sci U S A. 2002; 99(11): 7588-7593.
[34] Torres M, May R, Scharff M D, Casadevall A. Variable-region-identical antibodies differing in isotype demonstrate differences in fine specificity and idiotype. Journal of Immunology 2005; 174: 2132-2142.
[35] Beenhouwer D O, Yoo E M, Lai C W, Rocha M A, Morrison S L. Human immunoglobulin G2 (IgG2) and IgG4, but not IgG1 or IgG3, protect mice against Cryptococcus neoformans Infection. Infect Immun. March 2007; 75: 1424-1435.

1. An immunoglobulin preparation containing natural polyclonal IgG-reactive antibodies (Abs) isolated from human serum, for the treatment of HIV-1 infection.

2. The preparation of claim 1, which is obtained by the following steps:
(a) passing normal human serum (NHS) through a matrix specifically reactive with human IgG, particularly GammaBind G Sepharose, and recovering the matrix bound and the matrix-unbound fraction (“GammaBind G flow-through” or “GBF” fraction);
(b) eluting the matrix-bound IgG fraction obtained in (a), to obtain affinity purified IgG;
(c) covalent binding of the affinity-purified IgG obtained in (b), to CNBr-activated Sepharose 4B, or to an equivalent matrix, to obtain IgG-Sepharose;
(d) contacting the GBF fraction obtained in (a) with IgG-Sepharose at high ionic strength (equivalent to 5-fold concentrated PBS) and eluting the IgG-Sepharose bound Igs to obtain IgG-reactive (GBF) Abs.

3. The preparation of claim 2, further containing IgG-reactive IgG obtained by the following steps:
(a′) passing NHS through a matrix able to bind human IgG, particularly GammaBind G Sepharose matrix, and eluting the matrix-bound IgG fraction to obtain affinity purified IgG;
(b′) covalent binding of one aliquot of affinity purified IgG obtained in (a′), to CNBr-activated Sepharose 4B or an equivalent matrix, to obtain IgG-Sepharose;
(c′) contacting a second aliquot of affinity purified IgG obtained in (a′) with IgG-Sepharose obtained in (b′) at high ionic strength (equivalent to 5-fold concentrated PBS) and eluting IgG-Sepharose bound Igs to obtain IgG-reactive IgG.

4. The preparation of claim 2, wherein the IgG-reactive (GBF) Abs interact with B cell receptors, Fc receptors, MHC complex, T regulatory cell receptors, DC surface FcγRIIa.

5. The preparation of claim 1, which is enriched in IgG₂.

6. The preparation of claim 5, wherein said IgG₂ are IgG₂ dimers.

7. A pharmaceutical composition for the prophylaxis or treatment of HIV-1 infection, comprising an effective amount of the preparation of claim 1.

8. The pharmaceutical composition of claim 7, to be administered via subcutaneous, intramuscular or intravenous route.

9. The pharmaceutical composition of claim 8, to be administered as add-on therapy to antiretroviral therapy.

10. The pharmaceutical composition of claim 9, containing recombinant monoclional or polyclonal IgG-reactive IgG₂.

11. A method for preventing or treating HIV-1 infection, which comprises administering to a subject in need thereof, an immunoglobulin preparation according to claim 1.

12. The method of claim 11, wherein the subject has been exposed to HIV-1.

13. The method of claim 11, wherein the subject has been positively diagnosed for HIV-1 infection.

14. The method of claim 13, wherein said subject is under anti-retroviral therapy.

15. The preparation of claim 2, which is enriched in IgG₂.

16. The preparation of claim 3, which is enriched in IgG₂.

17. The preparation of claim 4, which is enriched in IgG₂.

18. A pharmaceutical composition for the prophylaxis or treatment of HIV-1 infection, comprising an effective amount of the preparation of claim 2.

19. A pharmaceutical composition for the prophylaxis or treatment of HIV-1 infection, comprising an effective amount of the preparation of claim 3.

20. A pharmaceutical composition for the prophylaxis or treatment of HIV-1 infection, comprising an effective amount of the preparation of claim 4.
